(19) United States
(12) Patent Application Publication
(10) Pub. No.: US 2018/0169057 A1
(43) Pub. Date: Jun. 21, 2018

(54) TREATMENT OF INFLAMMATORY LESIONS IN SUBJECTS AFFLICTED WITH MODERATE TO SEVERE ACNE

(71) Applicant: GALDERMA RESEARCH & DEVELOPMENT, BIOT (FR)

(72) Inventor: MARIE-LINE ABOU-CHACRA VERNET, NICE (FR)

(21) Appl. No.: 15/385,264

(22) Filed: Dec. 20, 2016

(51) Int. Cl. A61K 31/327; A61K 31/192; A61K 9/00; A61K 9/06; A61K 47/34; A61K 47/10; A61K 47/32

(52) U.S. Cl. CPC A61K 31/327; A61K 31/192; A61K 9/0014; A61K 47/32; A61K 47/34; A61K 47/10; A61K 9/06

(57) ABSTRACT

Provided is a regimen for therapeutic treatment of inflammatory acne lesions in a subject afflicted with moderate to severe acne. The regimen can include topically applying to said subject's skin, as active ingredients, 0.3% by weight adapalene and 2.5% by weight benzoyl peroxide, combined in a single formula that delivers said active ingredients together to achieve, in a group of such subjects, a decrease of at least about 80% in the number of said subjects having nodules and/or cysts after treatment with said regimen, wherein said percents by weight are relative to the weight of the single formula, and wherein said single formula is applied once or twice daily for a period of about 12 weeks.

### BACKGROUND

### Technical Field

The present application relates to the combined administration of 0.3% by weight adapalene and of 2.5% by weight benzoyl peroxide in a single formula for treatment of inflammatory lesions, including nodules and/or cysts in patients suffering from moderate to severe acne.

Description of Background and/or Related and/or Prior Art:

6-[3-(1-Adamantyl)-4-methoxyphenyl]-2-naphthoic acid (referred to hereinbelow as adapalene) is a naphthoic acid derivative with retinoid and anti-inflammatory properties. This molecule was the subject of development for the topical treatment of common acne and of dermatoses sensitive to retinoids.

Adapalene is marketed under the trademark Differin® at a weight concentration of 0.1%, in the form of an “alcoholic lotion” solution, an aqueous gel and a cream. These compositions are useful for treating acne. WO 03/075908 A1 describes adapalene compositions at a weight concentration of 0.3%, for treating acne.

WO 03/055472 moreover describes stable pharmaceutical compositions comprising adapalene and benzoyl peroxide (BPO) with pH independent gelling agents. Use of such compositions in synergistically treating acne lesions is described in WO 2008/006888 A1. An aqueous gel of 0.1% by weight adapalene and 2.5% by weight benzoyl peroxide is marketed under the trademark Epiduo®.

### SUMMARY

It has now surprisingly been demonstrated that a therapeutic combination of 0.3% by weight adapalene and 2.5% by weight benzoyl peroxide (BPO) can produce a degree of success and an improvement in the reduction of inflammatory lesions in patients afflicted with moderate to severe acne, particularly a percent decrease in the number of patients afflicted with various forms of acne, such as severe and/or moderately severe, including nodulocystic acne exhibiting one or more nodules and/or cysts, that is superior to a treatment based on a combination of adapalene 0.1% and BPO 2.5% or a vehicle gel, while at the same time maintaining the same, or substantially the same, level of skin tolerance.

Exemplary embodiments of the present disclosure thus feature a formulation of adapalene or a pharmaceutically acceptable salt thereof for the preparation of a pharmaceutical composition, at the set dose of 0.3% by weight, administered together with the set dose of 2.5% by weight benzoyl peroxide (BPO) in a single formula, for the treatment of moderate to severe acne, especially to produce a percent decrease in the number of subjects having inflammatory acne lesions. In exemplary embodiments, the percent decrease is demonstrated in subjects having nodules and/or cysts.

Acne is initially characterized by keratinization disorders, which are sometimes invisible to the naked eye. Visible acne lesions then develop, while the size of the sebaceous glands and the production of sebum increase.

Exemplary embodiments also concern acne lesions in subjects afflicted with moderate to severe acne. The term “acne lesions” means non-inflammatory lesions (open and closed comedones) and inflammatory lesions (papules, pustules, nodules and cysts) caused by acne. Preferably, the inflammatory lesions in moderate to severe acne patients are treated with a 0.3% adapalene/2.5% benzoyl peroxide single formula.

More preferably, the pharmaceutical composition is administered by daily or twice daily cutaneous topical application.

The term “adapalene salts” means the salts formed with a pharmaceutically acceptable base, especially mineral bases such as sodium hydroxide, potassium hydroxide and ammonia or organic bases such as lysine, arginine or N-methylglucamine. The term “adapalene salts” also means the salts formed with fatty amines such as dioctylamine and stearylamine.

The expression “combination of 0.3% by weight adapalene or salt thereof with 2.5% by weight benzoyl peroxide” means a single composition/single formula comprising both adapalene or salt thereof and benzoyl peroxide in the percents indicated, these being percents by weight with respect to the total weight of the single formula/composition.

The term “fixed combination” should be understood as meaning a combination whose active principles are combined at the specified fixed doses in the same vehicle (single formula) that delivers them together to the point or region of application. Preferably, the pharmaceutical composition in the form of a fixed combination is a gel; in this case, the two active principles are dispersed and intimately mixed, during the manufacture, in the same vehicle, which delivers them together during the application of the gel, in the treatment of severe acne.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The identifying terms “CD0271” and “CD1579,” included, for example, in certain Tables of the present disclosure, correspond to adapalene and BPO, respectively.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular and plural forms are interchangeable unless the context clearly dictates otherwise. As used herein, unless specifically indicated otherwise, the word “or” is used in the “inclusive” sense of “and/or” and not the “exclusive” sense of “either/or.”

The term “pharmaceutically acceptable medium” means a medium that is compatible with the skin, mucous membranes and the integuments.

The term “aqueous gel” means a composition containing, in an aqueous phase, a viscoelastic mass formed from colloidal suspensions (gelling agent).

The term “pH independent gelling agent” means a gelling agent capable of giving the composition a viscosity that is sufficient to keep the adapalene and the benzoyl peroxide in suspension, even under the influence of a variation of pH caused by the release of benzoic acid by the benzoyl peroxide.

The treatments have a variable duration, depending on the patient and the severity of his acne. The treatment period may thus run from several weeks to several months. A suitable treatment period or regimen is at least about four weeks, preferably from about 1 to about 6 months and more preferably a duration of about 8 weeks to about 3 months (about 12 weeks) is preferable, the duration of the treatment possibly being prolonged, if necessary, in patients afflicted with severe acne.

In a first aspect, there is provided herein a regimen for therapeutic treatment of inflammatory acne lesions in a subject afflicted with moderate to severe acne, said regimen comprising topically applying to said subject's skin, as active ingredients, 0.3% by weight adapalene and 2.5% by weight benzoyl peroxide, combined in a single formula that delivers said active ingredients together to achieve, in a group of such subjects, a decrease of at least about 80% in the number of said subjects having nodules and/or cysts after treatment with said regimen, wherein said percents by weight are relative to the weight of the single formula, and wherein said single formula is applied once or twice daily for a period of about 12 weeks.

In a second aspect, there is provided herein a regimen for therapeutic treatment of inflammatory acne lesions in a subject afflicted with moderate to severe acne, said regimen comprising topically applying to said subject's skin, as active ingredients, 0.3% by weight adapalene and 2.5% by weight benzoyl peroxide, combined in a single formula that delivers said active ingredients together to reduce the number of nodules and/or cysts, achieving a decrease of at least about 80% in the number of said subjects having nodules and/or cysts after treatment with said regimen, wherein said percents by weight are relative to the weight of the single formula, and wherein said single formula is applied once or twice daily for a period of about 12 weeks.

### BRIEF DESCRIPTION OF THE DRAWINGS

FIG. 1 is a schematic of the study disposition.

FIG. 2 is a bar graph showing the change in the number of subjects having one or two nodules, over time, comparing 0.3% adapalene/2.5% benzoyl peroxide gel, 0.1% adapalene/2.5% benzoyl peroxide (EPIDUO®) Gel and Vehicle Gel in the Intent-to-Treat (ITT) population (having moderate to severe acne; each subject having a maximum of two nodules).

FIG. 3 is a bar graph showing the change in the number of subjects having one or two nodules, over time, comparing 0.3% adapalene/2.5% benzoyl peroxide gel, 0.1% adapalene/2.5% benzoyl peroxide (EPIDUO®) Gel and Vehicle Gel in the Intent-to-Treat (ITT) population (having severe acne; each subject having a maximum of two nodules).

FIG. 4 is a graph of the mean incidence of erythema, over time, for each of the same three gels.

FIG. 5 is a graph of the mean incidence of scaling, over time, for each of the same three gels.

FIG. 6 is a graph of the mean incidence of dryness, over time, for each of the same three gels.

FIG. 7 is a graph of the mean incidence of stinging and burning, over time, for each of the same three gels.

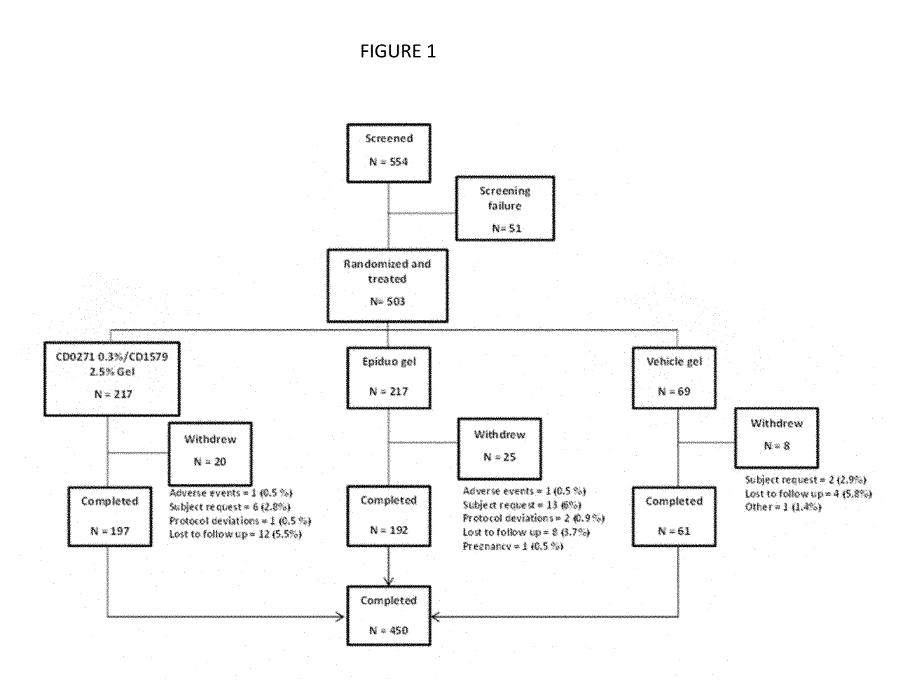

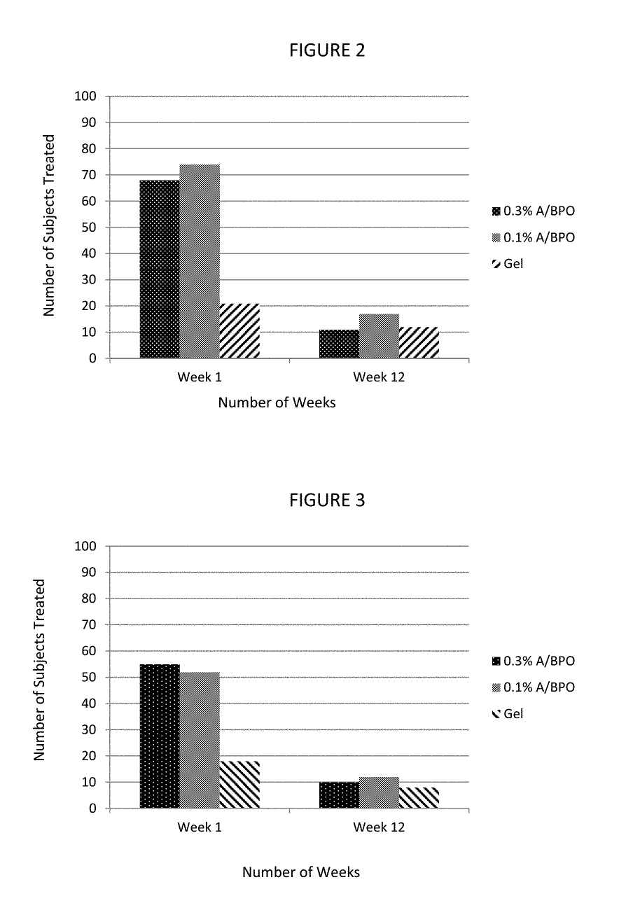

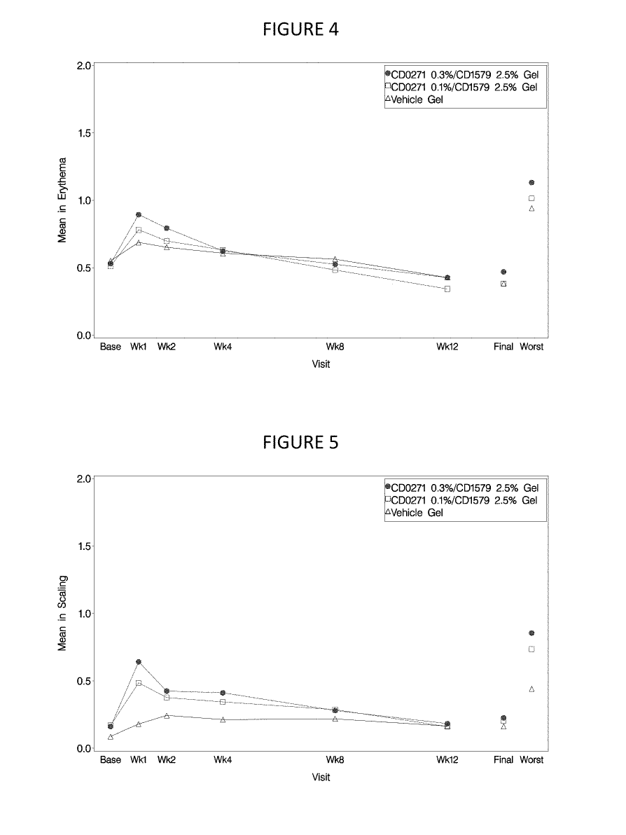

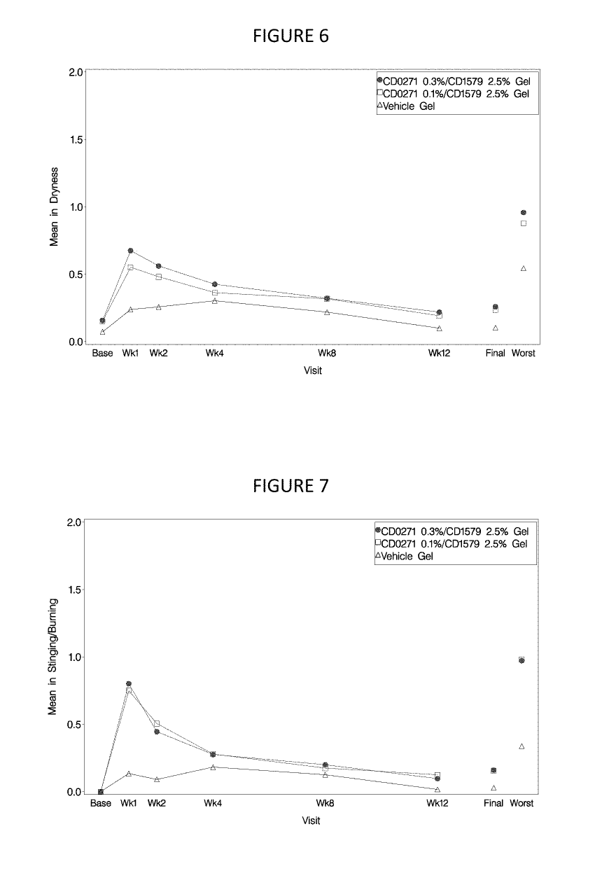

### DETAILED DESCRIPTION

In the first exemplary embodiment noted in the SUMMARY hereinabove, the following embodiments are noteworthy, separately or in combination:
(a) the inflammatory acne lesions are selected from nodules and cysts;
(b) the subject afflicted with moderate to severe acne has one or two nodules;
(c) the subject afflicted with moderate to severe acne has one or two cysts;
(d) the single formula is applied once daily;
(e) the single formula is an aqueous gel, a gel-cream, a cream or a lotion;
(f) the single formula is an aqueous gel;
(g) the aqueous gel comprises a pH independent gelling agent;
(h) the pH independent gelling agent is acrylamide/sodium acryloyldimethyl taurate copolymer and isohexadecane and polysorbate 80 (SIMULGEL 600) or polyacrylamide and isoparaffin and Laureth-7 (SEPIGEL 305);
(i) the pH independent gelling agent is SIMULGEL 600;
(j) the aqueous gel further comprises propylene glycol, glycerol and a poloxamer;
(k) the safety profile of said single formula is comparable to that observed under the same conditions for a comparative single formula comprising, as active ingredients, 0.1% by weight adapalene and 2.5% by weight benzoyl peroxide;
(l) the percent decrease in the number of said subjects having moderate to severe acne is from about 80% to about 86%;
(m) the percent decrease in the number of said subjects having severe acne is from about 80% to about 84%;
(n) the percent decrease in the number of said subjects having moderate to severe acne and treated with said single formula is at least about 20% higher than that observed under the same conditions for a vehicle; and
(o) the percent decrease in the number of said subjects having severe acne and treated with said single formula is at least about 20% higher than that observed under the same conditions for a vehicle.

In the second exemplary embodiment noted in the SUMMARY hereinabove, the following embodiments are noteworthy, separately or in combination:
(a) the subject afflicted with moderate to severe acne has one or two nodules;
(b) the subject afflicted with moderate to severe acne has one or two cysts;
(c) the single formula is applied once daily;
(d) the single formula is an aqueous gel, a gel-cream, a cream or a lotion;
(e) the single formula is an aqueous gel;
(f) the aqueous gel comprises a pH independent gelling agent;
(g) the pH independent gelling agent is acrylamide/sodium acryloyldimethyl taurate copolymer and isohexadecane and polysorbate 80 (SIMULGEL 600) or polyacrylamide and isoparaffin and Laureth-7 (SEPIGEL 305);
(h) the pH independent gelling agent is SIMULGEL 600;
(i) the aqueous gel further comprises propylene glycol, glycerol and a poloxamer;
(j) the safety profile of said single formula is comparable to that observed under the same conditions for a comparative single formula comprising, as active ingredients, 0.1% by weight adapalene and 2.5% by weight benzoyl peroxide;
(k) the percent decrease in the number of said subjects having moderate to severe acne is from about 80% to about 86%;
(l) the percent decrease in the number of said subjects having severe acne is from about 80% to about 84%;
(m) the percent decrease in the number of said subjects having moderate to severe acne and treated with said single formula is at least about 20% higher than that observed under the same conditions for a vehicle; and
(n) the percent decrease in the number of said subjects having severe acne and treated with said single formula is at least about 20% higher than that observed under the same conditions for a vehicle.

Exemplary pharmaceutical compositions/single formulas are particularly suited for topical treatment of the skin and the mucous membranes, and can be in the form of ointments, creams, milks, pomades, powders, impregnated pads, solutions, gels, gel-creams, sprays, lotions or suspensions. They can also be in the form of suspensions of microspheres or nanospheres or of lipid or polymeric vesicles, or of polymeric patches and hydrogels for controlled release. These compositions for topical application can be in anhydrous form, in aqueous form or in the form of an emulsion.

In a preferred embodiment, the pharmaceutical composition is in the form of a gel (in particular, an aqueous gel), a cream, a gel-cream or a lotion.

In a particular embodiment, the aqueous gel contains a pH independent gelling agent. Non-limiting examples of pH independent gelling agents that can be mentioned include the gelling agents of the polyacrylamide family, such as a mixture of sodium acryloyldimethyltaurate copolymer/isohexadecane/polysorbate 80 under the same SIMULGEL 600 by the company SEPPIC, the mixture of polyacrylamide/isoparaffin C13-12/laureth-7 such as, for example, the product sold under the name SEPIGEL 305 by the company SEPPIC, the family of acrylic polymers coupled to hydrophobic chains, such as the PEG-150/decyl/SMDI copolymer sold under the name ACULYN 44 (polycondensate comprising at least, as components, a polyethylene glycol containing 150 or 180 mol of ethylene oxide, decyl alcohol and methylenebis (4-cyclohexyl isocyanate) (SMDI), at 35% by weight in a mixture of propylene glycol (39%) and water (26%), the family of modified starches, such as the modified potato starch sold under the name Structure Solanace, or mixtures thereof.

The preferred gelling agents are derived from the polyacrylamide family, such as SIMULGEL 600 or SEPIGEL 305, or mixtures thereof.

The gelling agent as described above can be used in preferential concentrations ranging from about 0.1% to about 15% and more preferably ranging from about 0.5% to about 5%.

Alternatively, an aqueous gel can contain alternative or additional gelling agents such as carbomers (carbomer 940 or carbomer 980 or the like), if appropriate.

Exemplary single formula pharmaceutical compositions can also contain inert additives or combinations of these additives, such as one or more of the following:

wetting agents;

flavor enhancers;

preservatives such as para-hydroxybenzoic acid esters;

stabilizers;

moisture regulators;

pH regulators;

osmotic pressure modifiers;

emulsifiers;

UV-A and UV-B screening agents;

and antioxidants, such as α-tocopherol, butylhydroxyanisole or butylhydroxytoluene, superoxide dismutase, ubiquinol or certain metal chelating agents.

Of course, those skilled in the art will take care to select the optional compound(s) to be added to these compositions in such a way that the advantageous properties intrinsically associated with exemplary embodiments are not, or are not substantially, adversely affected by the envisaged addition.

The gel comprising 2.5% by weight benzoyl peroxide and 0.3% by weight adapalene and gelling agent, especially a pH independent gelling agent, advantageously comprises at least water and can also comprise a pro-penetrating agent and/or a liquid wetting surfactant.

The compositions can contain one or more pro-penetrating agents in preferential concentrations ranging from about 0% to about 20% and more preferably ranging from about 2% to about 6% by weight, relative to the total weight of the composition. They should generally not dissolve the active agents at the percentage used, should not cause any exothermic reactions harmful to the benzoyl peroxide, should aid in the satisfactory dispersion of the active agents, and should have antifoaming properties. Among the pro-penetrating agents preferably used, without this list being limiting, are compounds such as propylene glycol, dipropylene glycol, propylene glycol dipelargonate, lauroglycol and ethoxydiglycol.

A preferred pro-penetrating agent is propylene glycol.

Advantageously, exemplary compositions can also contain one or more liquid wetting surfactants in preferential concentrations ranging from about 0% to about 10% and more preferably ranging from about 0.1% to about 2%. The wetting power is the tendency of a liquid to spread over a surface.

They are preferably surfactants with an HLB (Hydrophilic-Lipophilic Balance) value from 7 to 9, or nonionic surfactants such as polyoxyethylenated and/or polyoxypropylenated copolymers. They should be liquid so as to be readily incorporated into the composition without it being necessary to heat them.

Among the wetting agents that are preferably used, without this list being limiting, are compounds of the Poloxamer family and more particularly Poloxamer 124 and/or Poloxamer 182.

The composition can also comprise any additive usually used in the cosmetics or pharmaceutical field, as noted previously, such as sequestering agents, antioxidants, sunscreens, preserving agents, fillers, electrolytes, humectants, colorants, common mineral or organic acids or bases, fragrances, essential oils, cosmetic active agents, moisturizers, vitamins, essential fatty acids, sphingolipids, self-tanning compounds such as DHA, and calmants and protective agents for the skin such as allantoin. Needless to say, a person skilled in the art will take care to select this or these optional additional compound(s), and/or the amount thereof, such that the advantageous properties of an exemplary composition are not, or are not substantially, adversely affected.

These additives can be present in the composition in a proportion of from about 0% to about 20% by weight relative to the total weight of the composition.

Examples of sequestering agents that can be mentioned include ethylenediaminetetraacetic acid (EDTA), and also derivatives or salts thereof, dihydroxyethylglycine, citric acid and tartaric acid, or mixtures thereof.

Examples of preserving agents that can be mentioned include benzalkonium chloride, phenoxyethanol, benzyl alcohol, diazolidinylurea and parabens, or mixtures thereof.

Examples of humectants that can be mentioned include glycerol and sorbitol.

Propylene glycol, glycerol and polyoxamer are particularly desirable as additives to the aqueous gels in which the gelling agent is SIMULGEL 600 or SEPIGEL 305 or other pH independent gelling agent.

An exemplary aqueous phase of the aqueous gel can comprise water, a floral water such as cornflower water, or natural mineral or spring water chosen, for example, from eau de Vittel, waters of the Vichy basin, eau d'Uriage, eau de la Roche Posay, eau de la Bourboule, eau d'Enghien-les-Bains, eau de Saint Gervais-les-Bains, eau de Neris-les-Bains, eau d'Allevard-les-Bains, eau de Digne, eau de Maizieres, eau de Neyrac-les-Bains, eau de Lons-le-Saunier, les Eaux Bonnes, eau de Rochefort, eau de Saint Christau, eau des Fumades, eau de Tercis-les-bains, eau d'Avene or eau d'Aix les Bains.

The aqueous phase can be present in a content of between about 10% and about 90% by weight and preferably between about 20% and about 80% by weight, relative to the total weight of the composition.

Exemplary single formula pharmaceutical compositions are especially intended for the treatment of moderate to severe acne, in particular, common acne (acne vulgaris), comedones, polymorphous acne, nodulocystic acne, acne conglobata, and secondary acne such as solar, drug-related or occupational acne. Moderate acne is acne having an [IGA]=3, where IGA is Investigative Global Assessment; and severe acne is acne having an [IGA]=4.

In order to further illustrate exemplary embodiments and the advantages thereof, the following specific Example is given, it being understood that same is intended only as illustrative, not limitative. In the Example to follow, the parts and percentages are given by weight, unless otherwise indicated.

### Example

In the clinical study and its summary which follows, the composition variously identified as “adapalene 0.3%/BPO 2.5% gel”, “adapalene 0.3%/benzoyl peroxide 2.5% topical gel” and “0.3% A/BPO” is an aqueous gel comprising the following, expressed as % by weight/total weight:

| | |
|---|---|
| Adapalene | 0.30% |
| Benzoyl peroxide | 2.50% |
| Copolymer of Acrylamide | 4.00% |
| and Sodium Acryloyldimethyl | |
| Taurate & Isohexadecane & | |
| Polysorbate 80 (SEPIGEL 600) | |
| Sodium docusate | 0.05% |
| Disodium EDTA | 0.10% |
| Glycerol | 4.00% |
| Poloxamer 124 | 0.20% |
| Propylene glycol | 4.00% |
| Purified water | qs 100%. |

The composition variously referred to in the clinical study and summary as “Epiduo Gel”, “adapalene 0.1%/BPO 2.5% gel”, “adapalene 0.1%/benzoyl peroxide 2.5% topical gel”, and “0.1% A/BPO” is an aqueous gel comprising the following, expressed as % by weight/total weight:

| | |
|---|---|
| Adapalene | 0.10% |
| Benzoyl peroxide | 2.50% |
| Copolymer of Acrylamide | 4.00% |
| and Sodium Acryloyldimethyl | |
| Taurate & Isohexadecane & | |
| Polysorbate 80 (SEPIGEL 600) | |
| Sodium docusate | 0.05% |
| Disodium EDTA | 0.10% |
| Glycerol | 4.00% |
| Poloxamer 124 | 0.20% |
| Propylene glycol | 4.00% |
| Purified water | qs 100%. |

The composition referred to in the clinical study and summary as “Vehicle Gel” or “vehicle” had the same composition as the two test gels described above, but excluded adapalene and benzoyl peroxide.

This study compared the efficacy and safety of adapalene 0.3%/benzoyl peroxide 2.5% (0.3% A/BPO) topical gel versus vehicle in subjects with moderate to severe acne (overall population [OP]), and in a subpopulation of the OP (severe acne subjects only) (severe population [SP]). The study also compared 0.3% A/BPO versus adapalene 0.1%/benzoyl peroxide 2.5% (0.1% A/BPO) topical gel in the SP.

This was a multicenter, randomized, double-blind, parallel-group, vehicle- and active-controlled study. As a screen failure rate of approximately 20% was expected, approximately 625 subjects were screened for a total of 497 subjects randomized/enrolled (in a 3:3:1 ratio for adapalene 0.3%/BPO 2.5% gel, Epiduo Gel, and Vehicle Gel, respectively).

A total of 554 subjects were screened and 503 subjects were randomized and included in the Intent-to-treat (ITT) population. All 503 randomized subjects were included in the safety population and 459 subjects (91.3%) were included in the per-protocol (PP) population. The distributions of subjects with baseline of moderate (IGA=3) and severe (IGA=4) acne were similar across treatment groups for each analysis population.

Key inclusion criteria for this controlled study were as follows:
male or female 12 years of age or older at screening visit;
clinical diagnosis of acne vulgaris with facial involvement;
an IGA of Moderate (3) or Severe (4) at screening and baseline visits;
a minimum of 20 but not more than 100 inflammatory lesions (papules and pustules) on the face (including the nose) at screening and baseline visits; and
a minimum of 30 but not more than 150 noninflammatory lesions (open comedones and closed comedones) on the face (including the nose) at screening and baseline visits.

Key exclusion criteria for this controlled study were as follows:
more than 2 acne nodules on the face at screening and baseline visits;
acne conglobata, acne fulminans, secondary acne (chloracne, drug-induced acne, etc.), nodulocystic acne, or acne requiring systemic treatment;
underlying diseases or other dermatologic conditions that require the use of interfering topical or systemic therapy or that might interfere with study assessments such as, but not limited to, atopic dermatitis, perioral dermatitis or rosacea. This includes clinically significant abnormal findings, uncontrolled or serious disease, or any medical or surgical condition, that may either interfere with the interpretation of the clinical trial results, and/or put the subject at significant risk (according to investigator's judgment) if he/she participates in the clinical trial;
the subject had received, applied or taken the specified treatments within the specified timeframe prior to the Baseline visit as described in the protocol;
the subject was unwilling to refrain from use of prohibited medication during the clinical trial;
use of hormonal contraceptives solely for control of acne; and
known or suspected allergies or sensitivities to any components of any of the study medications.

The efficacy of the studies described herein were assessed based on, but not limited to: (i) IGA, lesion counts; (ii) co-primary efficacy endpoints including success rate, i.e., the percentage of subjects with an IGA of clear or almost clear (and therefore at least a 2-grade improvement from baseline at week 12 [ITT]), the change in inflammatory lesion count from baseline to week 12 (ITT), and the change in noninflammatory lesion count from baseline to week 12 (ITT); and (iii) secondary efficacy endpoints, including percent changes in inflammatory and noninflammatory lesion counts from baseline to week 12 (ITT).

The safety of the studies described herein were assessed based on, but not limited to: (i) adverse events (AEs); (ii) local tolerability assessment; (iii) vital signs; (iv) physical examination; and (iii) urine pregnancy testing.

Hypothesis testing was in the form of hierarchical testing of the 3 treatment groups across and within the two severity strata (IGA=3 and IGA=4). The hierarchical test took place in steps, detailed below. At each step the co-primary endpoints were tested. It was required at each step that the test for each endpoint be significant at a two-tailed alpha level of 0.05.

The 3 successive steps in the testing of hypotheses were to compare:

1. Adapalene 0.3%/BPO 2.5% Gel versus Vehicle Gel (combined IGA severity strata [IGA 3 and 4]); if superiority at all 3 endpoints is shown, then:

2. Adapalene 0.3%/BPO 2.5% Gel versus Vehicle Gel within the severe (IGA=4) stratum; if superiority at all 3 endpoints was shown, then:

3. Adapalene 0.3%/BPO 2.5% Gel versus Epiduo Gel within the severe (IGA=4) stratum.

The difference between these active treatment groups was to be estimated via a 95% confidence interval. Due to the hierarchical testing structure, no adjustment for multiplicity was required. The statistical methodology for testing the Success Rate (proportion) was by Cochran-Mantel-Haenszel (CMH) test using general association. For Step 1, the test was stratified by IGA severity and analysis center; for Step 2, the test was stratified by analysis center; and for Step 3, the estimate of treatment difference did not use stratification. For all of the above-identified steps, the change in inflammatory and noninflammatory lesion counts from baseline to week 12 (ITT) was analyzed by analysis of covariance (ANCOVA) model including baseline lesion count as a covariate, analysis center and treatment as factor. For Step 1, baseline IGA severity was added to the model. The primary method of imputation for missing data used multiple imputation (MI) techniques, and the Last Observation Carried Forward (LOCF) was used for sensitivity analyses. The secondary analyses of Percent Change in Lesion Counts were performed using appropriate methodology.

All safety data were summarized based on the safety population, by baseline IGA and combined strata. Local tolerability variables (erythema, scaling, dryness, stinging/burning) were summarized by severity score and for worst response over time. Adverse events were tabulated in frequency tables by System Organ Class (SOC) and Preferred Term (PT) based on the Medical Dictionary for Regulatory Activities (MedDRA).

### Results: Demographics and Baseline Disease Characteristics

For the demographic data, no clinically meaningful differences were observed in the ITT population among the treatment groups. Gender distribution was balanced between males (47.7%) and females (52.3%) and the majority of subjects were white (77.3%). The mean age overall was 19.6 years (median 17.0 years), ranging from 12 to 57 years, and approximately half of the subjects (54.3%) were 12 to 17 years of age.

The data at baseline for skin phototype, IGA, and lesion counts showed no clinically meaningful differences among the treatment groups for any characteristics. Subjects representing the full range of skin phototypes (I to VI) were enrolled in each treatment group, with the majority having skin phototypes ranging from II through IV. For 251 subjects (49.9%), acne severity at baseline was moderate and 252 subjects (50.1%) had severe acne based on the IGA scale.

The mean baseline lesion counts in the overall population were: 38.2 inflammatory lesions, 59.6 noninflammatory lesions, and 98.2 total lesions. Nodule counts ranged from 0 to 2, consistent with the eligibility requirements for the study. Approximately half of the subjects (49.5%) had lesions on the trunk in addition to facial lesions.

For the severe stratum, no clinically meaningful differences were observed among the treatment groups for demographic or baseline characteristics. Gender distribution was balanced between males (47.6%) and females (52.4%), although the Vehicle Gel group had a higher percentage of males (61.8%) compared to the other groups, which had approximately the same proportions of male and female subjects. The majority of subjects were white (77.0%). Mean age and proportion of subjects 12 to 17 years of age were similar to the summary for combined severity strata (IGA=3 and IGA=4), and a full range of skin phototypes (I to VI) was represented in each treatment group. As expected, the mean baseline lesion counts were higher relative to the overall population (combined strata): 46.0 inflammatory lesions, 64.6 noninflammatory lesions, and 111.3 total lesions in in the severe stratum. In addition, a larger proportion of the subjects in the severe stratum (55.2%) had lesions on the trunk.

### Results: Efficacy

Efficacy was demonstrated for all three co-primary endpoints (Success Rate, Change from Baseline in Inflammatory Lesion Count and Change from Baseline in Noninflammatory Lesion Count) for Step 1 (analyses of co-primary endpoints in combined strata; Table 1 below).

TABLE 1

| | | | |
|---|---|---|---|
| | CD0271 0.3%/ | CD0271 0.1%/ | |
| | CD1579 2.5% | CD1579 2.5% | |
| | Gel | Gel | Vehicle Gel |
| Visit | (N = 217) | (N = 217) | (N = 69) |

Success Rate

| | | | |
|---|---|---|---|
| Week 12, MI | 33.7% | 27.3% | 11.0% |
| Difference from Vehicle^{a} | 22.7% | 16.3% | — |
| 95% CI^{a} | [12.8%, 32.6%] | [6.1%, 26.4%] | — |
| P-value vs. Vehicle^{b} | <0.001 | 0.014 | — |

Change from Baseline in Inflammatory Lesion Count

| | | | |
|---|---|---|---|
| Week 12, MI | | | |
| N (Change) | 217 | 217 | 69 |
| Mean (Change) | −27.79 | −26.46 | −13.19 |
| LS Mean Change from Baseline | −27.04 | −26.72 | −14.40 |
| SE (LS Mean) | 0.846 | 0.822 | 1.460 |
| LS Mean Difference from Vehicle | −12.84 | −12.32 | — |
| 95% CI^{c} | [−15.82, −9.46] | [−15.56, −9.07] | — |
| P-value vs. Vehicle^{c} | <0.001 | <0.001 | — |

Change from Baseline in Noninflammatory Lesion Count

| | | | |
|---|---|---|---|
| Week 12, MI | | | |
| N (Change) | 217 | 217 | 69 |
| Mean (Change) | −40.46 | −40.03 | −19.70 |
| LS Mean Change from Baseline | −40.18 | −39.00 | −18.47 |
| SE (LS Mean) | 1.332 | 1.277 | 2.270 |
| LS Mean Difference from Vehicle | −21.71 | −20.54 | — |
| 95% CI^{c} | [−26.66, −16.76] | [−25.59, −15.49] | — |
| P-value vs. Vehicle^{c} | <0.001 | <0.001 | — |

^{a}The method was based on ‘Combining Analysis Results from Multiply Imputed Categorical Data’ (Ra [symbol] 2013).
^{b}P-value vs. Vehicle, was from the Cochran-Mantel-Haenszel test stratified by Baseline IGA and analysis center with general association statistic using MI Methodology (Schafer 1997).
^{c}The 95% confidence interval and P-value vs. Vehicle were from the LS mean differences between each treatment group and vehicle using analysis of covariance (ANCOVA) with terms for treatment, baseline IGA severity, analysis center, and Baseline lesion count as covariate using MI Methodology.
CI = confidence interval;
LS = least squares;
SE = standard error [symbol]
indicates data missing or illegible when filed

In the severe stratum (IGA=4), analyses of co-primary endpoints for Adapalene 0.3%/BPO 2.5% Gel versus Vehicle Gel and versus Epiduo Gel are summarized in Table 2 below. All sensitivity analyses using the LOCF method at Week 12 showed similar results. The PP analyses for Success Rate and Changes in Inflammatory and Noninflammatory Lesion Counts in combined severity strata confirmed the conclusion from the primary analyses.

TABLE 2

| | | | |
|---|---|---|---|
| | CD0271 0.3%/ | CD0271 0.1%/ | |
| | CD1579 2.5% | CD1579 2.5% | |
| | Gel | Gel | Vehicle Gel |
| Visit | (N = 106) | (N = 112) | (N = 34) |

Success Rate

| | | | |
|---|---|---|---|
| Week 12, MI | 31.9% | 20.5% | 11.8% |
| Difference from Vehicle^{a} | 20.1% | 8.8% | — |
| 95% CI^{a} | [6.0%, 34.2%] | [−4.6%, 22.1%] | — |
| P-value vs. Vehicle^{b} | 0.029 | 0.443 | — |
| Difference from Epiduo | 11.4% | — | — |
| 95% CI^{a} | [−0.5%, 23.2%] | — | — |

Change from Baseline in inflammatory Lesion Count

| | | | |
|---|---|---|---|
| Week 12, MI | | | |
| N (Change) | 106 | 112 | 34 |
| Mean (Change) | −37.25 | −30.24 | −14.28 |
| LS Mean Change from Baseline | −35.17 | −31.92 | −15.46 |
| SE (LS Mean) | 1.407 | 1.320 | 2.297 |
| LS Mean Difference from Vehicle | −19.71 | −16.46 | — |
| 95% CI^{c} | [−24.81, −14.62] | [−21.70, −11.22] | — |
| P-value vs. Vehicle^{c} | <0.001 | <0.001 | — |
| LS Mean Difference from Epiduo | −3.25 | — | — |
| 95% CI^{c} | [−7.15, 0.64] | — | — |

Change from Baseline in Noninflammatory Lesion Count

| | | | |
|---|---|---|---|
| Week 12, MI | | | |
| N (Change) | 106 | 112 | 34 |
| Mean (Change) | −48.33 | −43.89 | −17.82 |
| LS Mean Change from Baseline | −45.61 | −43.10 | −17.25 |
| SE (LS Mean) | 2.058 | 1.847 | 3.337 |
| LS Mean Difference from Vehicle | −28.36 | −25.85 | — |
| 95% CI^{c} | [−35.64, −21.08] | [−33.42, −18.28] | — |
| P-value vs. Vehicle^{c} | <0.001 | <0.001 | — |
| LS Mean Difference from Epiduo | −2.51 | — | — |
| 95% CI^{c} | [−8.08, −3.06] | — | — |

^{a}The method was based on ‘Combining Analysis Results from Multiply Imputed Categorical Data’ (Ra [symbol] 2013).
^{b}P-value vs. Vehicle, was from the Cochran-Mantel-Haenszel test stratified by analysis center with general association statistic using MI Methodology (Schafer 1997).
^{c}The 95% confidence interval and P-value vs. Vehicle were from the LS mean differences between each treatment group and vehicle using analysis of covariance (ANCOVA) with terms for treatment, analysis center, and Baseline lesion count as covariate using MI Methodology.
CI = confidence interval;
LS = least squares;
SE = standard error [symbol]
indicates data missing or illegible when filed

For analyses of secondary endpoints (Percent Change in Inflammatory Lesion Count and Percent Change in Noninflammatory Lesion Count), control of multiplicity linked to the two secondary endpoints was achieved by the use of the Hochberg procedure. The sensitivity analyses using the LOCF method at Week 12 showed similar results.

Both Strata Combined (IGA=3 and IGA=4)
a. Adapalene 0.3%/BPO 2.5% Gel was statistically more effective than Vehicle Gel (p<0.001; Week 12, MI) based on the Hochberg adjustment. The mean percent change in inflammatory lesion counts from baseline to week 12 was −68.70% in the Adapalene 0.3%/BPO 2.5% Gel group versus only −39.23% in the Vehicle Gel group.
b. Adapalene 0.3%/BPO 2.5% Gel was statistically more effective than Vehicle Gel (p<0.001; Week 12, MI). The mean percent change in noninflammatory lesion counts from baseline to week 12 was −68.34% in the Adapalene 0.3%/BPO 2.5% Gel group versus only −37.38% in the Vehicle Gel group.

Severe Stratum (IGA=4)
a.—Adapalene 0.3%/BPO 2.5% Gel was statistically more effective than Vehicle Gel (p<0.001; Week 12, MI) based on the Hochberg adjustment. The mean percent change in inflammatory lesion counts from baseline to week 12 was −74.44% in the Adapalene 0.3%/BPO 2.5% Gel group versus only −33.00% in the Vehicle Gel group.
b. Adapalene 0.3%/BPO 2.5% Gel was statistically more effective than Vehicle Gel (p<0.001; Week 12, MI). The mean percent change in noninflammatory lesion counts from baseline to week 12 was −72.05% in the Adapalene 0.3%/BPO 2.5% Gel group versus only −30.79% in the Vehicle Gel group.

Statistical summary of the number of nodules at baseline for ITT, severe and moderate strata are presented in Table 3 below. As shown in Table 3, in the severe stratum, 50.4% of subjects had no nodules, 26.6% had 1, and 23% had 2 nodules.

TABLE 3

| Adapalene 0.3%/BPO 2.5% | | Adapalene 0.1%/BPO 2.5% | | Gel vehicle | | All | |
|---|---|---|---|---|---|---|---|
| N | % | N | % | N | % | N | % |

All Subjects

| | | | | | | | | |
|---|---|---|---|---|---|---|---|---|
| Baseline value | 149 | 68.7 | 143 | 65.9 | 48 | 69.5 | 340 | 67.6 |
| 0 | | | | | | | | |
| 1 | 42 | 19.4 | 49 | 22.6 | 12 | 17.4 | 103 | 20.5 |
| 2 | 26 | 12.0 | 25 | 11.5 | 9 | 13.0 | 60 | 11.9 |
| All | 217 | 100.0 | 217 | 100.0 | 69 | 100.0 | 503 | 100.0 |

Severe stratum

| | | | | | | | | |
|---|---|---|---|---|---|---|---|---|
| Baseline value | 51 | 48.1 | 60 | 53.6 | 16 | 47.1 | 127 | 50.4 |
| 0 | | | | | | | | |
| 1 | 30 | 28.3 | 28 | 25.3 | 9 | 26.5 | 67 | 26.6 |
| 2 | 25 | 23.8 | 24 | 21.4 | 9 | 26.5 | 58 | 23.0 |
| All | 106 | 100.0 | 112 | 100.0 | 34 | 100.0 | 252 | 100.0 |

Moderate stratum

| | | | | | | | | |
|---|---|---|---|---|---|---|---|---|
| Baseline value | 98 | 88.3 | 83 | 79.0 | 32 | 91.4 | 213 | 84.9 |
| 0 | | | | | | | | |
| 1 | 12 | 10.8 | 21 | 20.0 | 3 | 8.6 | 36 | 14.3 |
| 2 | 1 | 0.9 | 1 | 1.0 | . | . | 2 | 0.8 |
| All | 111 | 100.0 | 105 | 100.0 | 35 | 100.0 | 251 | 100.0 |

Table 4 below summarizes the changes (shift) in the number of nodules from baseline to week 12 for the overall, severe, and moderate acne populations. As shown in Table 4, after 12 weeks of treatment, the mean number of nodules decreased in the active treatment groups. A greater decrease is observed in the two active groups compared with the gel vehicle group. In the severe acne subgroup, the trend appeared to be dose-related. Shift tables for nodules between baseline and week 12 showed that the percentage of subjects with no nodules at week 12 increased with the dose of adapalene in the fixed-dose combination (0.3% vs. 0.1% vs. vehicle), in both the combined subject population and the subgroup of subjects with severe acne, irrespective of the number of nodules at baseline.

TABLE 4

| Week 12 (LOCF) | | | | | | | | | | | | | | | |
|---|---|---|---|---|---|---|---|---|---|---|---|---|---|---|---|
| Adapalene 0.3%/BPO 2.5% | | | | | | Adapalene 0.1%/BPO 2.5% | | | | | | | | | |
| 0 | | 1 | | All | | 0 | | 1 | | 2 | | 3 | | All | |
| N | % | N | % | N | % | N | % | N | % | N | % | N | % | N | % |

ITT

| | | | | | | | | | | | | | | | | |
|---|---|---|---|---|---|---|---|---|---|---|---|---|---|---|---|---|
| Baseline Value | 148 | 99.3 | 1 | 0.7 | 149 | 100.0 | 139 | 97.2 | 3 | 2.1 | . | . | 1 | 0.7 | 143 | 100.0 |
| 0 | | | | | | | | | | | | | | | | |
| 1 | 36 | 85.7 | 6 | 14.3 | 42 | 100.0 | 39 | 79.6 | 9 | 18.4 | 1 | 2.0 | . | . | 49 | 100.0 |
| 2 | 21 | 80.8 | 5 | 19.2 | 26 | 100.0 | 18 | 72.0 | 4 | 16.0 | 1 | 4.0 | 2 | 8.0 | 25 | 100.0 |
| All | 205 | 94.5 | 12 | 5.5 | 217 | 100.0 | 196 | 90.3 | 16 | 7.4 | 2 | 0.9 | 3 | 1.4 | 217 | 100.0 |

Severe stratum

| | | | | | | | | | | | | | | | | |
|---|---|---|---|---|---|---|---|---|---|---|---|---|---|---|---|---|
| Baseline Value | 51 | 100.0 | . | . | 51 | 100.0 | 59 | 98.3 | 1 | 1.7 | . | . | . | . | 60 | 100.0 |
| 0 | | | | | | | | | | | | | | | | |
| 1 | 25 | 83.3 | 5 | 16.7 | 30 | 100.0 | 23 | 82.1 | 4 | 14.3 | 1 | 3.6 | . | . | 28 | 100.0 |
| 2 | 20 | 80.0 | 5 | 20.0 | 25 | 100.0 | 17 | 70.8 | 4 | 16.7 | 1 | 4.2 | 2 | 8.3 | 24 | 100.0 |
| All | 96 | 90.6 | 10 | 9.4 | 106 | 100.0 | 99 | 88.4 | 9 | 8.0 | 2 | 1.8 | 2 | 1.8 | 112 | 100.0 |

Moderate stratum

| | | | | | | | | | | | | | | | | |
|---|---|---|---|---|---|---|---|---|---|---|---|---|---|---|---|---|
| Baseline Value | 97 | 99.0 | 1 | 1.0 | 98 | 100.0 | 80 | 98.4 | 2 | 2.4 | . | . | 1 | 1.2 | 83 | 100.0 |
| 0 | | | | | | | | | | | | | | | | |
| 1 | 11 | 91.7 | 1 | 8.3 | 12 | 100.0 | 16 | 76.2 | 5 | 23.8 | . | . | . | . | 21 | 100.0 |
| 2 | 1 | 100.0 | . | . | 1 | 100.0 | 1 | 100.0 | . | . | . | . | . | . | 1 | 100.0 |
| All | 109 | 982 | 2 | 1.8 | 111 | 100.0 | 97 | 92.4 | 7 | 6.7 | . | . | 1 | 1.0 | 105 | 100.0 |

| | Week 12 (LOCF) | | | | | | | | | |
|---|---|---|---|---|---|---|---|---|---|---|
| | Topical Gel Vehicle | | | | | | | | | |
| | 0 | | 1 | | 2 | | 4 | | All | |
| | N | % | N | % | N | % | N | % | N | % |
| ITT | | | | | | | | | | |
| Baseline Value | 42 | 87.5 | 4 | 8.3 | 1 | 2.1 | 1 | 2.1 | 48 | 100.0 |
| 0 | | | | | | | | | | |
| 1 | 8 | 66.7 | 2 | 16.7 | 2 | 16.7 | . | . | 12 | 100.0 |
| 2 | 4 | 44.4 | 4 | 44.4 | 1 | 11.1 | . | . | 9 | 100.0 |
| All | 54 | 78.3 | 10 | 14.5 | 4 | 5.8 | 1 | 1.4 | 69 | 100.0 |
| Severe stratum | | | | | | | | | | |
| Baseline Value | 12 | 75.0 | 2 | 12.5 | 1 | 6.3 | 1 | 6.3 | 16 | 100.0 |
| 0 | | | | | | | | | | |
| 1 | 6 | 66.7 | 1 | 11.1 | 2 | 22.2 | . | . | 9 | 100.0 |
| 2 | 4 | 44.4 | 4 | 44.4 | 1 | 11.1 | . | . | 9 | 100.0 |
| All | 22 | 64.7 | 7 | 20.6 | 4 | 11.8 | 1 | 2.9 | 34 | 100.0 |
| Moderate stratum | | | | | | | | | | |
| Baseline Value | 30 | 93.8 | 2 | 6.3 | . | . | . | . | 32 | 100.0 |
| 0 | | | | | | | | | | |
| 1 | 2 | 66.7 | 1 | 33.3 | . | . | . | . | 3 | 100.0 |
| 2 | . | . | . | . | . | . | . | . | . | . |
| All | 32 | 91.4 | 3 | 8.6 | . | . | . | . | 35 | 100.0 |

A statistical analysis of the total lesion counts at baseline in this study in the adapalene 0.3%/BPO 2.5% arm are shown in Table 5 below.

TABLE 5

| | CD0271 0.3%/ | CD0271 0.1%/ | | |
|---|---|---|---|---|
| | CD1579 2.5% Gel | CD1579 2.5% Gel | Vehicle Gel | Total |
| Variable | (N = 106) | (N = 112) | (N = 34) | (N = 252) |
| Baseline Inflammatory Lesion Count | | | | |
| N | 106 | 112 | 34 | 252 |
| Mean | 49.0 | 43.7 | 44.5 | 46.0 |
| S.D. | 20.18 | 17.24 | 19.32 | 18.90 |
| Min, Max | 21, 99 | 20, 99 | 20, 99 | 20, 99 |
| Baseline Non-Inflammatory Lesion Count | | | | |
| N | 106 | 112 | 34 | 252 |
| Mean | 64.4 | 64.7 | 64.8 | 64.6 |
| S.D. | 27.77 | 33.64 | 30.61 | 30.76 |
| Min, Max | 30, 147 | 30, 149 | 30, 138 | 30, 149 |
| Baseline Total Lesion Count | | | | |
| N | 106 | 112 | 34 | 252 |
| Mean | 114.1 | 109.0 | 110.1 | 111.3 |
| S.D. | 41.31 | 44.36 | 40.09 | 42.44 |
| Min, Max | 54, 226 | 54, 221 | 52, 202 | 52, 226 |
| Baseline Nodules | | | | |
| N | 106 | 112 | 34 | 252 |
| Mean | 0.8 | 0.7 | 0.8 | 0.7 |
| S.D. | 0.81 | 0.81 | 0.84 | 0.81 |
| Min, Max | 0, 2 | 0, 2 | 0, 2 | 0, 2 |
| Any Baseline Lesions on Trunk? | | | | |
| Yes | 65 (61.3%) | 56 (50.0%) | 18 (52.9%) | 139 (55.2%) |
| No | 41 (38.7%) | 56 (50.0%) | 16 (47.1%) | 113 (44.8%) |
| Total | 106 (100.0%) | 112 (100.0%) | 34 (100.0%) | 252 (100.0%) |

[symbol]

[symbol]

indicates data missing or illegible when filed

Based on the type of lesions (papules and pustules), number of lesions, and severity based on body areas involved (61.3% of severe subjects had truncal lesions), the subjects in the adapalene 0.3%/BPO 2.5% arm were indeed representative of the severe stratum and not just the lower range of severe acne vulgaris. This conclusion is in alignment with the Kligman-Plewig scale, which is based on the type, number, and severity of lesions (Table 6 below).

TABLE 6

| Algorithm of the | | | | | |
|---|---|---|---|---|---|
| German Acne | | | | | |
| Guideline | Kiloman/Plewia | | | Leeds Scale | |
| — | — | | | No acne | 0 |
| Light | Acne comedonica | Grade I | <10 comedos | Acne minor | 0.25 |
| | | Grade II | 10-25 comedos | | 0.5 |
| | | Grade III | 26-50 comedos | | 0.75 |
| | | Grade IV | >50 comedos | | 1.25 |
| Moderately severe | Acne papulopustulosa | Grade I | <10 Papules/Pustules | Acne major | 1.5 |
| (papulopustulosa) | | Gade II | 10-20 Papules/Pustules | | 1.75 |
| | | Grade III | 21-30 Papules/Pustules | | 2 |
| | | Grade IV | >30 Papules/Pustules | | 3 |
| Severe | Acne congiobate | | | | 5 |
| | | | | | 8 |

As the line extension of Adapalene 0.1%/BPO 2.5% Gel, Adapalene 0.3%/BPO 2.5% Gel is considered to extend the continuum of care in the treatment of acne severity spectrum. At present, there is no topical therapy, which spares the use of oral antibiotics, approved for this type of severity, which the Applicant considers an unmet medical need.

### Quality of Life Assessments (Combined Strata):

At week 12, although the mean DLQI scores were similar across treatment groups, a larger proportion of subjects in the Adapalene 0.3%/BPO 2.5% Gel group (57.8%) perceived that their acne had no effect on their quality of life in comparison to the Vehicle Gel group (25.0%).

At baseline and week 12, the mean CDLQI scores were similar in each treatment group, and the distributions of score ranges (i.e., no effect, small effect, etc.) showed no apparent differences among treatment groups.

At week 12, the mean EQ-5D-3L scores did not show any noticeable change from baseline in any group.

In the overall study population, significant differences were observed (p<0.001) between treatment groups for Adapalene 0.3%/BPO 2.5% Gel relative to Vehicle Gel. Most subjects reported moderate (36.3%), marked (44.6%), or complete improvement (9.8%) in their acne at week 12 in the Adapalene 0.3%/BPO 2.5% Gel group relative to the Vehicle Gel group (20.0%, 15.4%, and 4.6%, respectively), with median scores of 1.0 and 3.0 in each respective group.

In the severe stratum, significant differences were observed (p<0.001) between treatment groups for Subject's Assessment of Acne for Adapalene 0.3%/BPO 2.5% Gel relative to Vehicle Gel. At week 12, significant differences were observed (p<0.001) between treatment groups for Adapalene 0.3%/BPO 2.5% Gel relative to Vehicle Gel. Most subjects reported moderate (35.1%), marked (50.5%), or complete improvement (8.2%) in their acne at week 12 in the Adapalene 0.3%/BPO 2.5% Gel group relative to the Vehicle Gel group (22.6%, 9.7%, and 6.5%, respectively), with median scores of 1.0 and 3.0 in each respective group.

The data from the Appreciation Questionnaire at week 12 showed that more subjects in the Adapalene 0.3%/BPO 2.5% Gel group had a favorable impression concerning the use and the texture of the study drug than subjects in the Vehicle Gel group, most notably for Question 1 (I am satisfied with the product): 71.6% agreed versus 32.3% in the Vehicle Gel group. Higher proportions of subjects in the Adapalene 0.3%/BPO 2.5% Gel and Epiduo Gel groups viewed the study drug less favorably for Question 2 (The product irritates my skin) compared to the Vehicle Gel group (Agree=7.4% and 3.1%, respectively). The distributions of responses for all questions were generally similar between the Adapalene 0.3%/BPO 2.5% Gel and Epiduo Gel groups.

### Safety in ITT Population:

A total of 50 subjects (23.0%) in the Adapalene 0.3%/BPO 2.5% Gel, 42 subjects (19.4%) in the Epiduo Gel, and 13 subjects (18.8%) in the Vehicle Gel group reported AEs during the study (Table 7 below).

TABLE 7

| | CD0271 | CD0271 | | |
|---|---|---|---|---|
| | 0.3%/ | 0.1%/ | | |
| | CD1579 | CD1579 | | |
| | 2.5% Gel | 2.5% Gel | Vehicle Gel | Total |
| Category | (N = 217) | (N = 217) | (N = 68) | (N = 503) |
| Subjects With At Least One Adverse Event | 50 (23.0%) | 42 (19.4%) | 13 (18.8%) | 105 (20.9%) |
| Related AE | 12 (5.5%) | 1 (0.5%) | 0 | 13 (2.6%) |
| Unrelated AE | 41 (18.9%) | 41 (18.9%) | 13 (18.8%) | 95 (18.9%) |
| Subjects With At Least One Serious Adverse Event | 0 | 1 (0.5%) | 0 | 1 (0.2%) |
| Unrelated AE | 0 | 1 (0.5%) | 0 | 1 (0.2%) |
| Subjects With At Least One Adverse Event Leading to | 1 (0.5%) | 1 (0.5%) | 0 | 2 (0.4%) |
| Discontinuation | | | | |
| Related AE | 1 (0.5%) | 0 | 0 | 1 (0.2%) |
| Unrelated AE | 0 | 1 (0.5%) | 0 | 1 (0.2%) |
| Subjects With At Least One Mild Adverse Event | 44 (20.3%) | 33 (15.2%) | 9 (13.0%) | 86 (17.1%) |
| Related AE | 7 (3.2%) | 1 (0.5%) | 0 | 8 (1.6%) |
| Unrelated AE | 37 (17.1%) | 32 (14.7%) | 9 (13.0%) | 78 (15.5%) |
| Subjects With At Least One Moderate Adverse Event | 15 (6.9%) | 17 (7.8%) | 5 (7.2%) | 37 (7.4%) |
| Related AE | 6 (2.8%) | 0 | 0 | 6 (1.2%) |
| Unrelated AE | 10 (4.6%) | 17 (7.8%) | 5 (7.2%) | 32 (6.4%) |
| Subjects With At Least One Severe Adverse Event | 0 | 0 | 1 (1.4%) | 1 (0.2%) |
| Unreleted AE | 0 | 0 | 1 (1.4%) | 1 (0.2%) |
| Subjects With At Least One Adverse Event of Special | 1 (0.5%) | 0 | 0 | 1 (0.2%) |
| Interest | | | | |
| Related AE | 1 (0.5%) | 0 | 0 | 1 (0.2%) |

Subjects might be counted twice, once in Related AE category and once in Unrelated AE category for having more than one AE.

In the Adapalene 0.3%/BPO 2.5% Gel group, 12 subjects (5.5%) had AEs that were assessed by Investigators as related to study drug, compared to 1 subject (0.5%) in the Epiduo Gel and 0 subjects in the Vehicle Gel group (Table 8 below).

TABLE 8

| | CD0271 | CD0271 | | |
|---|---|---|---|---|
| | 0.3%/ | 0.1%/ | | |
| | CD1579 | CD1579 | | |
| | 2.5% Gel | 2.5% Gel | Vehicle Gel | Total |
| System Organ Class/Preferred Term² | (N = 217) | (N = 217) | (N = 69) | (N = 503) |
| Total Number of AE(s) | 15 | 2 | 0 | 17 |
| Total Number (%) of Subjects with AE(s)^{b} | 12 (5.5%) | 1 (0.5%) | 0 | 13 (2.6%) |
| Skin and subcutaneous tissue disorders | 11 (5.1%) | 1 (0.5%) | 0 | 12 (2.4%) |
| Skin irritation | 6 (2.8%) | 0 | 0 | 6 (1.2%) |
| Pruritus | 1 (0.5%) | 1 (0.5%) | 0 | 2 (0.4%) |
| Skin burning sensation | 2 (0.9%) | 0 | 0 | 2 (0.4%) |
| Dermatitis atopic | 1 (0.5%) | 0 | 0 | 1 (0.2%) |
| Eczema | 1 (0.5%) | 0 | 0 | 1 (0.2%) |
| Erythema | 0 | 1 (0.5%) | 0 | 1 (0.2%) |
| Rash | 1 (0.5%) | 0 | 0 | 1 (0.2%) |
| Skin hypopigmentation | 1 (0.5%) | 0 | 0 | 1 (0.2%) |
| Eye disorders | 1 (0.5%) | 0 | 0 | 1 (0.2%) |
| Erythema of eyelid | 1 (0.5%) | 0 | 0 | 1 (0.2%) |
| Nervous system disorders | 1 (0.5%) | 0 | 0 | 1 (0.2%) |
| Paraesthesia | 1 (0.5%) | 0 | 0 | 1 (0.2%) |

^{a}A subject was counted only once for multiple occurrences within a System Organ Class or Preferred Term.
^{b}A subject was counted once even if the subject experienced more than one adverse event during the study.

In the Adapalene 0.3%/BPO 2.5% Gel group, the most common AEs were nasopharyngitis (6.5% of subjects) and skin irritation (4.1%). In the Epiduo Gel group, the most common AEs were nasopharyngitis (5.1% of subjects), upper respiratory tract infection (2.3%), and allergic dermatis (1.4%). In the Vehicle Gel group, upper respiratory tract infection was the only AE reported in more than 1 subject (4 subjects [5.8%]). Most AEs that were assessed by the Investigators as related to study drug were in the Skin and Subcutaneous Tissue Disorders SOC. Most of the related AEs occurred in subjects in the Adapalene 0.3%/BPO 2.5% Gel group (15 AEs in 12 subjects) and the remaining 2 related AEs occurred in 1 subject in the Epiduo Gel group. In the Adapalene 0.3%/BPO 2.5% Gel group, the most common related AEs were skin irritation (2.8% of subjects) and skin burning sensation (0.9%). In the Adapalene 0.3%/BPO 2.5% Gel group, 1 subject had a related AE of atopic dermatitis that led to the subject's discontinuation from the study and 1 subject had a related AESI of mild skin irritation.

Most AEs reported in this study were of either mild or moderate severity. Only 1 subject was reported to have a severe AE (hypokalemia), in the Vehicle Gel group, and the AE was assessed by the Investigator as unrelated to study drug.

No deaths were reported in this study. The only SAE in the study was an unrelated SAE of generalized anxiety disorder reported for a subject in the Epiduo Gel group.

Two subjects discontinued due to AEs: 1 subject in the Adapalene 0.3%/BPO 2.5% Gel had an AE of atopic dermatitis assessed as moderate and related to study drug and 1 subject in the Epiduo Gel group had worsening of acne assessed as moderate and not related to study drug.

Two (2) subjects reported pregnancies: 1 in the Epiduo Gel group and 1 in the Vehicle Gel group. Both pregnancies were ongoing at the time of last report.

Sensitization was suspected for 1 subject (AESI of mild skin irritation), but was neither confirmed nor ruled out because the subject did not consent to patch testing.

Overall, signs/symptoms for local tolerability parameters (erythema, scaling, dryness, stinging/burning) for highest score worse than Baseline were more common in the Adapalene 0.3%/BPO 2.5% Gel group than in the Epiduo Gel and Vehicle Gel groups. However, mean scores tended to decrease over time and severity in the Adapalene 0.3%/BPO 2.5% Gel group was mostly mild or moderate, with few subjects (6.1%) experiencing severe signs/symptoms.

No clinically meaningful abnormal trends were observed in vital signs or physical examinations in any treatment group.

### Safety in Severe Stratum:

An overall summary of AEs in the Severe stratum, including relationship to study drug as assessed by the Investigators is provided in Table 9 below. A total of 21 subjects (19.8%) in the Adapalene 0.3%/BPO 2.5% Gel, 17 subjects (15.2%) in the Epiduo Gel, and 7 subjects (20.6%) in the Vehicle Gel group had AEs during the study. In the Adapalene 0.3%/BPO 2.5% Gel group, 2 (1.9%) of the subjects had AEs assessed by Investigators as related to study drug, and 0 subjects in the other groups.

In the Adapalene 0.3%/BPO 2.5% Gel group, the most common AEs were nasopharyngitis (7.5% of subjects), influenza (1.9%), and skin irritation (1.9%). In the Epiduo Gel group, the most common AEs were nasopharyngitis (4.5% of subjects), upper respiratory tract infection (1.8%), and allergic dermatis (1.8%). In the Vehicle Gel group, no AEs were observed in more than 1 subject (2.9%).

The most common AE in the Skin and Subcutaneous Tissue Disorders SOC was skin irritation (1.9%), which was observed in 1 subject (0.9%) in the Epiduo Gel group and was not observed in the Vehicle Gel group. Allergic dermatis was reported in 2 subjects (1.8%) in the Epiduo Gel group and was not observed in the other groups. The incidence of related AEs was 1.9% (2 subjects) in the Adapalene 0.3%/BPO 2.5% Gel group and related AEs were not observed in the other groups. The related AEs were atopic dermatitis (AE leading to discontinuation) and skin irritation.

TABLE 9

| | CD0271 | CD0271 | | |
|---|---|---|---|---|
| | 0.3%/ | 0.1%/ | | |
| | CD1579 | CD1579 | | |
| | 2.5% Gel | 2.5% Gel | Vehicle Gel | Total |
| Category | (N = 106) | (N = 112) | (N = 34) | (N = 252) |
| Subjects With At Least One Adverse Event | 21 (19.8%) | 17 (15.2%) | 7 (20.6%) | 45 (17.9%) |
| Related AE | 2 (1.9%) | 0 | 0 | 2 (0.8%) |
| Unrelated AE | 19 (17.9%) | 17 (15.2%) | 7 (20.6%) | 43 (17.1%) |
| Subjects With At Least One Serious Adverse Event | 0 | 1 (0.9%) | 0 | 1 (0.4%) |
| Unrelated AE | 0 | 1 (0.9%) | 0 | 1 (0.4%) |
| Subjects With At Least One Adverse Event Leading to | 1 (0.9%) | 1 (0.9%) | 0 | 2 (0.8%) |
| Discontinuation | | | | |
| Related AE | 1 (0.9%) | 0 | 0 | 1 (0.4%) |
| Unrelated AE | 0 | 1 (0.9%) | 0 | 1 (0.4%) |
| Subjects With At Least One Mild Adverse Event | 17 (16.0%) | 12 (10.7%) | 5 (14.7%) | 34 (13.5%) |
| Related AE | 1 (0.9%) | 0 | 0 | 1 (0.4%) |
| Unrelated AE | 16 (15.1%) | 12 (10.7%) | 5 (14.7%) | 33 (13.1%) |
| Subjects With At Least One Moderate Adverse Event | 8 (7.5%) | 9 (8.0%) | 3 (8.8%) | 20 (7.9%) |
| Related AE | 1 (0.9%) | 0 | 0 | 1 (0.4%) |
| Unrelated AE | 7 (6.6%) | 9 (8.0%) | 3 (8.8%) | 19 (7.5%) |
| Subjects With At Least One Severe Adverse Event | 0 | 0 | 1 (2.9%) | 1 (0.4%) |
| Unrelated AE | 0 | 0 | 1 (2.9%) | 1 (0.4%) |
| Subjects With At Least One Adverse Event of Special | 0 | 0 | 0 | 0 |
| Interest | | | | |

Subjects might be counted twice, once in Related AE category and once in Unrelated AE category for having more than one AE.

The subject who had the SAE of generalized anxiety disorder (Epiduo Gel group), the 2 subjects in the Adapalene 0.3%/BPO 2.5% Gel group who discontinued due to AEs (atopic dermatitis and worsening acne), and the subject with the severe AE (hypokalemia) in the Vehicle Gel group were all in the severe stratum.

No AESIs were observed in the severe stratum.

Similar to the trend observed in the overall study population, signs/symptoms for local tolerability parameters in the severe stratum for highest score worse than baseline were more common among subjects in the Adapalene 0.3%/BPO 2.5% Gel group than in the Epiduo Gel and Vehicle Gel groups and likewise mean scores tended to decrease over time. Severity in the Adapalene 0.3%/BPO 2.5% Gel group was mostly mild or moderate with few subjects (4.9%) experiencing severe signs/symptoms.

This study demonstrated that Adapalene 0.3%/BPO 2.5% Gel was superior to vehicle in the co-primary endpoints of IGA success rate (clear/almost clear and at least 2-grade change) and changes in inflammatory and noninflammatory lesion counts. The statistical significance was consistently high (p<0.001) and the outcome clinically compelling. The study is robust, presenting consistent results in the PP population and sensitivity analyses.

In the subgroup of subjects with severe acne (baseline IGA=4), Adapalene 0.3%/BPO 2.5% Gel was superior to Vehicle in IGA success rate (p=0.029), and changes in inflammatory (p<0.001) and noninflammatory lesion counts (p<0.001), with Success Rate reflecting at least a 3-grade improvement in IGA scores during the 12-week treatment period. The outcome was supported by robust sensitivity analyses.

A trend of numerical superiority of Adapalene 0.3%/BPO 2.5% Gel compared to Epiduo Gel in IGA Success Rate and Changes in Inflammatory and Noninflammatory Lesion Counts was observed in subjects with severe acne (IGA=4).

Adapalene 0.3%/BPO 2.5% Gel was well-tolerated in this study. Adverse events (AEs) that were treatment emergent were generally mild to moderate in severity. The few TEAEs that were related to treatment were generally dermatological in nature, mild to moderate in severity, and did not require discontinuation of treatment.

Thus, the study provides confirmatory evidence of the efficacy and safety of Adapalene 0.3%/BPO 2.5% Gel in the target population.

While exemplary embodiments have been described in terms of various specific and preferred embodiments, the skilled artisan will appreciate that various modifications, substitutions, omissions, and changes can be made without departing from the spirit thereof. Accordingly, it is intended that the scope of the application be limited solely by the scope of the following claims.

1. A regimen for therapeutic treatment of inflammatory acne lesions in a subject afflicted with moderate to severe acne and having one or two nodules and/or cysts, said regimen comprising topically applying to said subject's skin, as active ingredients, 0.3% by weight adapalene and 2.5% by weight benzoyl peroxide, combined in a single formula, wherein said percents by weight are relative to the weight of the single formula, that delivers said active ingredients together to achieve complete suppression of the nodules and/or cysts with an 80% success rate, wherein said single formula is applied once or twice daily for a period of about 12 weeks, and wherein a safety profile of said single formula is comparable to that observed after treatment under the same conditions with a comparative single formula comprising, as active ingredients, 0.1% by weight adapalene and 2.5% by weight benzoyl peroxide.

2. (canceled)

3. (canceled)

4. The regimen according to claim 1, wherein said single formula is applied once daily.

5. The regimen according to claim 1, wherein said single formula is an aqueous gel, a gel-cream, a cream or a lotion.

6. The regimen according to claim 1, wherein said single formula is an aqueous gel.

7. The regimen according to claim 6, wherein said aqueous gel comprises a pH independent gelling agent.

8. The regimen according to claim 7, wherein said pH independent gelling agent is acrylamide/sodium acryloyldimethyl taurate copolymer and isohexadecane and polysorbate 80 (SIMULGEL 600) or polyacrylamide and isoparaffin and Laureth-7 (SEPIGEL 305).

9. The regimen according to claim 8, wherein said pH independent gelling agent is SIMULGEL 600.

10. The regimen according to claim 7, wherein said aqueous gel further comprises propylene glycol, glycerol and a poloxamer.

11. (canceled)

12. The regimen according to claim 1, wherein a subject having moderate to severe acne has from about 80% to about 86% success rate of completely reducing the number of nodules and/or cysts.

13. The regimen according to claim 1, wherein a subject having severe acne has from about 80% to about 84% success rate of completely reducing the number of nodules and/or cysts.

14. The regimen according to claim 1, wherein a subject having moderate to severe acne and treated with said single formula has at least about 20% higher success rate of completely reducing the number of nodules and/or cysts than that observed after treatment under the same conditions with a vehicle alone.

15. The regimen according to claim 1, wherein a subject having severe acne and treated with said single formula has at least about 20% higher success rate of completely reducing the number of nodules and/or cysts than that observed after treatment under the same conditions with a vehicle alone.

16. A regimen for therapeutic treatment of inflammatory acne lesions in a subject afflicted with nodulocystic acne and having one or two nodules and/or cysts, said regimen comprising topically applying to said subject's skin, as active ingredients, 0.3% by weight adapalene and 2.5% by weight benzoyl peroxide, combined in a single formula, wherein said percents by weight are relative to the weight of the single formula, that delivers said active ingredients together to completely reduce the number of nodules and/or cysts with an 80% success rate, wherein said single formula is applied once or twice daily for a period of about 12 weeks, and wherein a safety profile of said single formula is comparable to that observed after treatment under the same conditions with a comparative single formula comprising, as active ingredients, 0.1% by weight adapalene and 2.5% by weight benzoyl peroxide.

17. (canceled)

18. (canceled)

19. The regimen according to claim 16, wherein said single formula is applied once daily.

20. The regimen according to claim 16, wherein said single formula is an aqueous gel, a gel-cream, a cream or a lotion.

21. The regimen according to claim 16, wherein said single formula is an aqueous gel.

22. The regimen according to claim 21, wherein said aqueous gel comprises a pH independent gelling agent.

23. The regimen according to claim 22, wherein said pH independent gelling agent is acrylamide/sodium acryloyldimethyl taurate copolymer and isohexadecane and polysorbate 80 (SIMULGEL 600) or polyacrylamide and isoparaffin and Laureth-7 (SEPIGEL 305).

24. The regimen according to claim 23, wherein said pH independent gelling agent is SIMULGEL 600.

25. The regimen according to claim 22, wherein said aqueous gel further comprises propylene glycol, glycerol and a poloxamer.

26. (canceled)

28. The regimen according to claim 17, wherein a subject having moderate to severe acne has from about 80% to about 86% success rate of completely reducing the number of nodules and/or cysts.

29. The regimen according to claim 17, wherein a subject having severe acne is from about 80% to about 84% success rate of completely reducing the number of nodules and/or cysts.

30. The regimen according to claim 17, wherein a subject having moderate to severe acne and treated with said single formula has at least about 20% higher success rate of completely reducing the number of nodules and/or cysts than that observed after treatment under the same conditions with a vehicle alone.

31. The regimen according to claim 17, wherein a subject having severe acne and treated with said single formula has at least about 20% higher success rate of completely reducing the number of nodules and/or cysts than that observed after treatment under the same conditions with a vehicle alone.
